(19) United States
(12) Patent Application Publication
(10) Pub. No.: US 2025/0213818 A1
(43) Pub. Date: Jul. 3, 2025

(54) S-Shape Catheter

(71) Applicant: Ihab Bassam Alomari, Anaheim, CA (US)

(72) Inventors: Ihab Bassam Alomari, Anaheim, CA (US); Islam Abudayyeh, Redlands, CA (US); Steven Burstein, Los Angeles, CA (US)

(73) Assignee: Ihab Bassam Alomari, Anaheim, CA (US)

(21) Appl. No.: 18/200,577

(22) Filed: May 23, 2023

(51) Int. Cl. A61M 25/01; A61M 25/00

(52) U.S. Cl. CPC A61M 25/0105; A61M 25/0054

(57) ABSTRACT

The present application provides a method inserting a S-Shape catheter to pulmonary artery of a person in need. Generally, the S-Shape catheter can be introduced through femoral vein or inferior vena cava; and then advanced through right atrium, right ventricle, and right ventricular outflow tract; and then advanced to the pulmonary artery through pulmonary valve.

### BACKGROUND

Pulmonary artery catheter (PAC) (also called Swan-Ganz catheter) is extensively used to monitor the heart's function and blood flow and pressures in and around the heart. Often, PAC is inserted into the right side of the heart and the arteries leading to the lungs by passing a thin tube (catheter) through a variety of veins around a human body. For example, PAC can be inserted intravascularly through a central vein, such as the femoral, jugular, antecubital or brachial vein, to connect to the right side of the heart and advanced into the pulmonary artery.

Typical route for insertion of PAC includes introducing the catheter through one of the main central veins (e.g., subclavian, internal jugular, femoral); the catheter traversing into the superior or inferior vena cava, and then reaching the right atrium. Subsequently, PAC reaches the right ventricle through the tricuspid valve. From there, the PAC is advanced into the right ventricular outflow tract and eventually to the pulmonary artery through the pulmonary valve.

If the distal tip of PAC is equipped with a balloon, the balloon can be inflated and deflated after reaching pulmonary artery to take measurement of critical cardiac parameters, such as pulmonary capillary wedge (or occlusive) pressure which gives an indirect assessment of left sided filling pressures. The PAC is also suitable for delivery of a stent or guide wire to the pulmonary artery. Other suitable devices or pharmaceutics/biologics, which can be attached to the distal end of PAC, include anticoagulation agent, thrombolytics, vasodilators and other infusions.

Because of these advantages, the PAC remains an excellent tool to obtain critical cardiac readings for medical practitioners to diagnose cardiac diseases or make relevant treatment decisions. With an inserted PAC, it is possible to measure right atrial, pulmonary artery, and pulmonary capillary wedge pressures, and to determine cardiac output and oxygen saturations in the right heart chambers. Other possible applications of PAC include monitoring mixed venous oxygen saturation, intra-cardiac pacing and assessing of right-ventricular volumes, ejection fraction, pulmonary hypertension, cardiogenic shock, unexplained dyspnea, right-sided cardiac chamber filling pressures, intracardiac shunts, cardiac valves, or vascular resistance. Therefore, PAC is widely used in the management of acute myocardial infarction and high-risk cardiac and noncardiac surgical patients.

Even though insertion of PAC is generally a safe procedure to perform, there are occasional risks or complications associated with such operation. Typical risks or complications include cardiac tamponade, heart attack, injury to a coronary artery, irregular heartbeat, low blood pressure, stroke, bleeding, infection, and pain at the IV or sheath insertion site, damage to the blood vessels, and blood clots.

### SUMMARY OF THE INVENTION

In one aspect, the present invention provides a method inserting a S-Shape catheter to pulmonary artery of a person in need. The method comprises: introducing the catheter through femoral vein or inferior vena cava; advancing the catheter through right atrium, right ventricle, and right ventricular outflow tract; and advancing the catheter to the pulmonary artery through pulmonary valve. In the method, the S-Shape catheter comprises: a proximal end and a distal end and a tube extending from the proximal end to the distal end of the catheter. The tube of the catheter further comprises: a first curve and a secondary curve disposed between the proximal end and the distal end, and an intermediate section disposed between the first curve and the second curve. The first curve of the tube is located proximate to the proximal end; and the secondary curve of the tube is located distal to the first curve and disposed proximate to the distal end. The secondary curve is configured in an opposite direction to the first curve. Furthermore, the first curve has a first inner radius, a first arc angle, and a first arc length; and the second curve has a second inner radius, a second arc angle, and a second arc length.

In certain embodiments, the first inner radius is from about 0.10 cm to about 10.0 cm. In some preferred embodiments, the first inner radius is from about 1.0 cm to about 5.0 cm.

In certain embodiments, the first arc angle is from about 10 degrees to about 170 degrees. In some preferred embodiments, the first arc angle is from about 30 degrees to about 150 degrees.

In certain embodiments, the first arc length is from about 0.10 cm to about 30.0 cm. In some preferred embodiments, the first arc length is from about 0.75 cm to about 20.0 cm.

In certain embodiments, the second inner radius is from about 0.10 cm to about 10.0 cm. In some preferred embodiments, the second inner radius is from about 0.5 cm to about 5.0 cm.

In certain embodiments, the second arc angle is from about 10 degrees to about 170 degrees. In some preferred embodiments, the second arc angle is from about 30 degrees to about 150 degrees.

In certain embodiments, the second arc length is from about 0.10 cm to about 30.0 cm. In some preferred embodiments, the second arc length is from about 0.20 cm to about 20.0 cm.

In further embodiments, the distal end of the catheter is attached with a functional device. In certain embodiments, the functional device may be a medical balloon, a stent, balloon tip, end-hole catheter, closed-tip catheter, or a combination thereof.

In certain embodiments, the S-Shape catheter is either pre-manufactured or is formed by a medical practitioner before the operation of inserting the catheter.

### BRIEF DESCRIPTION OF DRAWINGS

FIG. 1 depicts a schematic view of an S-Shape pulmonary catheter having a proximal end and a distal end, and a tube extending from the proximal end to the distal end, where the tube is divided (from the proximal end to the distal end) into a proximal section 150, a first curve 110, an intermediate section 160; a secondary curve 120, a distal section 170, and a catheter tip 180. In the figure, 100—S-Shape Catheter; 110 (N1)—First Curve; 120 (N2)—Secondary Curve; 150—Proximal Section; 160—Intermediate Section; 170—Distal Section; 180—Catheter Tip; R1—First Inner Radius; θ1—First Arc Angle; AL1—First Arc Length; R2—Second Inner Radius; θ2—Second Arc Angle; and AL2—Second Arc Length.

FIG. 2 illustrates an example of S-Shape pulmonary catheter.

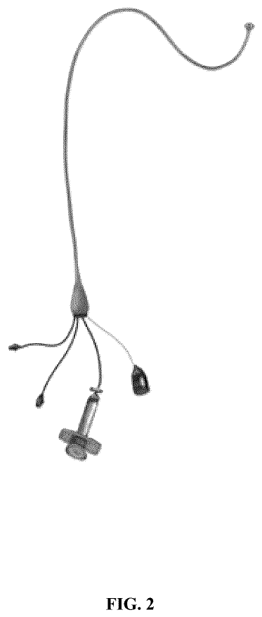

FIG. 3 illustrates a detailed drawing of right atrium, right ventricle, pulmonary artery, and relative position of the inserted pulmonary artery catheter and method inserting the catheter to its designed position. In the figure, N1 and N2 are the primary and the secondary curves, respectively.

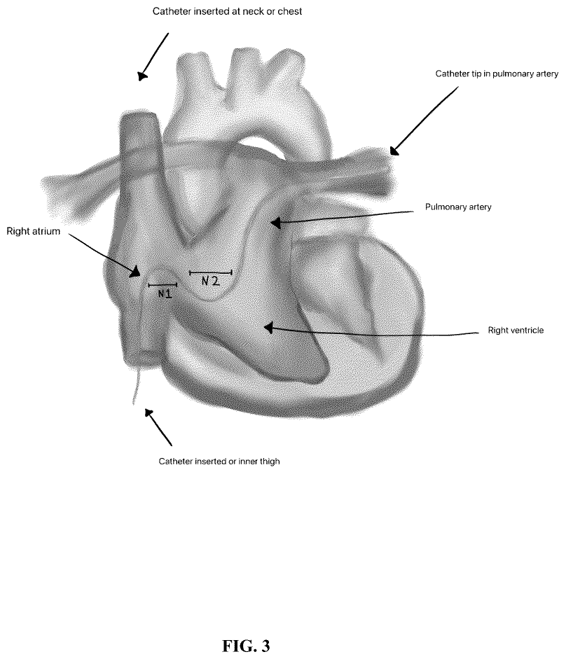

### DETAILED DESCRIPTION OF INVENTION

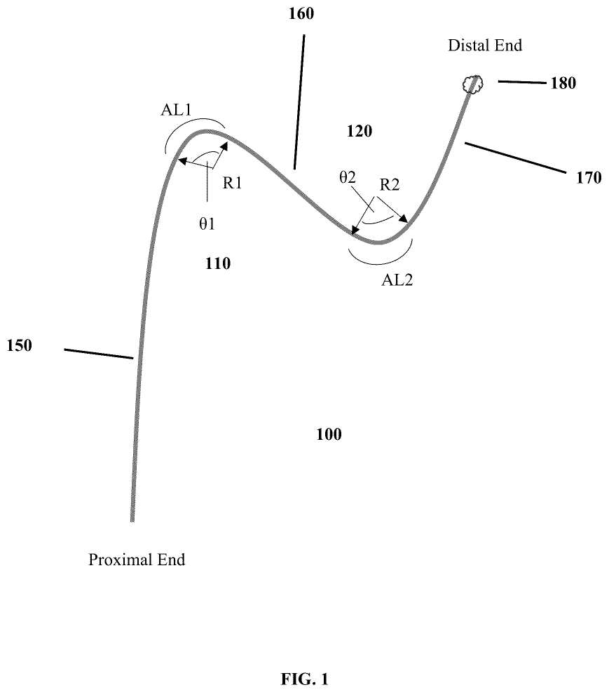

In one aspect, the instant disclosure provides a catheter adapted to be inserted into pulmonary artery of a living body. More particularly, the catheter has an S-Shape tube (FIG. 1) for insertion to pulmonary artery through femoral vein or inferior vena cava for providing easier and more precise access to the pulmonary artery. The disclosed catheter is hereinafter called S-Shape catheter, S-Shape catheter for pulmonary artery, or S-Shape pulmonary artery catheter (S-Shape PAC).

Currently, PAC are primarily introduced through the jugular vein in the neck. This requires obtaining access through a puncture in the internal jugular vein in the neck and is both inconvenient and can be uncomfortable for the patient. However, it is a trend now to advance those catheters through the femoral vein in the leg. This is less uncomfortable, more convenient to perform in the cardiac laboratory, and easier to perform for the physician. The typical design of current PAC has a C-shape to the distal end of it making advancing it from the leg and up through the heart to the lungs challenging. The C-shape designs have only one curve of variable diameters and will often coil in the atrium and the right ventricle, also when advancing it from the groin, the catheter tip will point toward the heart apex, which is opposite direction to the pulmonary artery, therefore, presents difficulty crossing up into pulmonary arteries. Operators then can spend a significant amount of time trying to advance the catheter into position increasing radiation exposure, and possibly increasing procedural risk.

In this disclosure, an S-shape catheter for pulmonary artery insertion is provided. The S-Shape of instant PAC allows it to cross up to the pulmonary artery from femoral vein in the leg much more easily and offer better control of the PAC during the operation.

In one embodiment, the S-shape PAC comprises a proximal, and a distal end, and a tube extending from the proximal end to the distal end, where the tube is divided (from the proximal end to the distal end) into a proximal section 150, a first curve 110, an intermediate section 160; a secondary curve 120, a distal section 170, and a catheter tip 180. (FIG. 1).

In the same embodiment, the proximal section of the tube 150 locates at the proximal end of the catheter and is situated between the proximal end of catheter and the first curve 110. The first curve 110 is situated between the proximal section 150 and the intermediate section 160.

In the same embodiment, the intermediate section 160 is situated between the first curve 110 and the secondary curve 120. first curve

In the same embodiment, the secondary curve 120 is situated between the intermediate section 160 and the distal section 170.

In the same embodiment, the distal section 170 is situated between the secondary curve 120 and the distal end of the catheter. The tube at the distal end of the catheter is attached to a catheter tip. The catheter tip comprises a functional device, which includes a medical balloon, a stent, balloon tip, end-hole catheter, closed-tip catheter, or a combination thereof.

In the same embodiment, the first curve is defined by a first inner radius R1, a first arc angle θ1, and a first arc length AL1. Similarly, the secondary curve is defined by a second inner radius R2, a second arc angle θ2, and a second arc length AL2

In certain embodiments, the first inner radius R1 and the second inner radius R2 independently range from about 0.1 cm to about 10 cm. In certain embodiment, the first inner radius R1 and the second inner radius R2 independently are about 0.10 cm, about 0.15 cm, about 0.20 cm, about 0.25 cm, about 0.30 cm, about 0.35 cm, about 0.40 cm, about 0.45 cm, about 0.50 cm, about 0.55 cm, about 0.60 cm, about 0.65 cm, about 0.70 cm, about 0.75 cm, about 0.80 cm, about 0.85 cm, about 0.90 cm, about 0.95 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.2 cm, about 2.4 cm, about 2.6 cm, about 2.8 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, or about 10.0 cm.

In a preferred embodiment, the first inner radius R1 ranges from about 1.0 cm to about 5.0 cm. In the embodiment, the first inner radius R1 is, for example, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.5 cm, about 3.0 cm, about 3.5 cm, about 4.0 cm, about 4.5 cm, or about 5.0 cm.

In another preferred embodiment, the second inner radius R2 ranges from about 0.5 cm to about 5.0 cm. In the embodiment, the second inner radius R2 is, for example, about 0.50 cm, about 0.55 cm, about 0.60 cm, about 0.65 cm, about 0.70 cm, about 0.75 cm, about 0.80 cm, about 0.85 cm, about 0.90 cm, about 0.95 cm, about 1.0 cm, about 1.1 cm, about 1.2 cm, about 1.3 cm, about 1.4 cm, about 1.5 cm, about 1.6 cm, about 1.7 cm, about 1.8 cm, about 1.9 cm, about 2.0 cm, about 2.5 cm, about 3.0 cm, about 3.5 cm, about 4.0 cm, about 4.5 cm, or about 5.0 cm.

In certain embodiments, the first arc angle θ1 and the second arc angle θ2 independently range from about 10° to about 170°. In certain embodiments, the first arc angle θ1 and the second arc angle θ2 independently are about 10°, about 11°, about 12°, about 13°, about 14°, about 15°, about 16°, about 17°, about 18°, about 19°, about 20°, about 25°, about 30°, about 35°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, about 150°, about 155°, about 160°, about 165°, or about 170°.

In a preferred embodiment, the first arc angle θ1 and the second arc angle θ2 independently range from about 30° to about 150°. In the embodiment, the first arc angle θ1 and the second arc angle θ2 independently are, for example, about 30°, about 31°, about 32°, about 33°, about 34°, about 35°, about 36°, about 37°, about 38°, about 39°, about 40°, about 45°, about 50°, about 55°, about 60°, about 65°, about 70°, about 75°, about 80°, about 85°, about 90°, about 95°, about 100°, about 105°, about 110°, about 115°, about 120°, about 125°, about 130°, about 135°, about 140°, about 145°, or about 150°.

In certain embodiments, the first arc length AL1 and the second arc length AL2 independently range from about 0.10 cm to about 30.00 cm. In certain embodiments, the first arc length AL1 and the second arc length AL2 independently are about 0.10 cm, about 0.15 cm, about 0.20 cm, about 0.25 cm, about 0.30 cm, about 0.35 cm, about 0.40 cm, about 0.45 cm, about 0.50 cm, about 0.55 cm, about 0.60 cm, about 0.65 cm, about 0.70 cm, about 0.75 cm, about 0.80 cm, about 0.85 cm, about 0.90 cm, about 0.95 cm, about 1.0 cm, about 1.5 cm, about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 11.0 cm, about 12.0 cm, about 13.0 cm, about 14.0 cm, about 15.0 cm, about 16.0 cm, about 17.0 cm, about 18.0 cm, about 19.0 cm, about 20.0 cm, about 21.0 cm, about 22.0 cm, about 23.0 cm, about 24.0 cm, about 25.0 cm, about 26.0 cm, about 27.0 cm, about 28.0 cm, about 29.0 cm, or about 30.0 cm.

In a preferred embodiment, the first arc length AL1 ranges from about 0.75 cm to about 20.0 cm. In the embodiment, the first arc length AL1 is, for example, about 0.75 cm, about 0.80 cm, about 0.85 cm, about 0.90 cm, about 0.95 cm, about 1.0 cm, about 1.5 cm, about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 11.0 cm, about 12.0 cm, about 13.0 cm, about 14.0 cm, about 15.0 cm, about 16.0 cm, about 17.0 cm, about 18.0 cm, about 19.0 cm, or about 20.0 cm.

In another preferred embodiment, the second arc length AL2 ranges from about 0.20 cm to about 20.00 cm. In the embodiment, the second arc length AL2 is, for example, about 0.20 cm, about 0.25 cm, about 0.30 cm, about 0.35 cm, about 0.40 cm, about 0.45 cm, about 0.50 cm, about 0.55 cm, about 0.60 cm, about 0.65 cm, about 0.70 cm, about 0.75 cm, about 0.80 cm, about 0.85 cm, about 0.90 cm, about 0.95 cm, about 1.0 cm, about 1.5 cm, about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, about 8.0 cm, about 8.5 cm, about 9.0 cm, about 9.5 cm, about 10.0 cm, about 11.0 cm, about 12.0 cm, about 13.0 cm, about 14.0 cm, about 15.0 cm, about 16.0 cm, about 17.0 cm, about 18.0 cm, about 19.0 cm, or about 20.0 cm.

In certain embodiments, the proximal end of the S-Shape catheter may be equipped with suitable medical devices. Examples of suitable medical devices may include syringe, infusion port, luer lock port.

In the above embodiments, the S-Shape catheter is pre-manufactured or is formed by a practitioner in medical or relevant fields before the operation to insert the catheter to pulmonary artery of a person in need.

In another aspect, the instant disclosure provides a method of inserting the S-Shape catheter to pulmonary artery of a person in need. The method of inserting the S-Shape catheter comprises: introducing the catheter through femoral vein or inferior vena cava; advancing the catheter through right atrium, right ventricle, and right ventricular outflow tract; and advancing the catheter to the pulmonary artery through pulmonary valve. The procedure can be done by first inserting a sheath into the femoral vein. Under fluoroscopic guidance, the balloon tip is inflated and the catheter is advanced up the inferior vena cava to the right atrium. From there, the operator manipulated the catheter across the tricuspid valve, into the right ventricle, and up the pulmonary artery across the pulmonic valve.

Hereinafter, the present invention will be described in more detail by way of Examples. The invention will be more readily understood by reference to the following examples. However, the technical scope of the present invention is not intended to be limited only to the following Examples.

### EXAMPLES

### Example 1. Dimensions of an S-Shape Pulmonary Artery Catheter

S-Shape PACs' suitability for pulmonary artery insertion were tested. The dimensional variables of the S-Shaped PACs fall within the preferred ranges as shown in the following table.

TABLE 1
Preferred Ranges of R1, θ1, AL1, R2,
θ2, and AL2 of S-Shape PACs

| | Preferred Ranges | |
|---|---|---|
| R1 | 1.0-5.0 | cm |
| θ1 | 30-150 | degrees |
| AL1 | 0.75-20.0 | cm |
| R2 | 0.5-5.0 | cm |
| θ2 | 30-150 | degrees |
| AL2 | 0.20-20.0 | cm |

### Example 2. Operation to Insert an S-Shape Catheter Through Femoral Vein or Inferior Vena Cava to Pulmonary Artery

The S-Shape PACs of Example 1 were tested in both heathy individuals and those having preexisting heart conditions. Specifically, the S-Shape PACs of Example 1 were introduced through femoral vein or inferior vena cava. Subsequently, they were advanced through right atrium, right ventricle, and right ventricular outflow tract; and were advanced to the pulmonary artery through pulmonary valve. More specifically, the procedure could be done by first inserting a sheath into the femoral vein. Under fluoroscopic guidance, the balloon tip of the PAC was inflated and the PAC was advanced up the inferior vena cava to the right atrium. From there, the operator manipulated the catheter across the tricuspid valve, into the right ventricle, and up the pulmonary artery across the pulmonic valve.

What is claimed is:

1. A method inserting a S-Shape catheter to pulmonary artery of a person in need, comprising:
introducing the catheter through femoral vein or inferior vena cava;
advancing the catheter through right atrium, right ventricle, and right ventricular outflow tract;
advancing the catheter to the pulmonary artery through pulmonary valve; and
wherein the S-Shape catheter comprises:
a proximal end and a distal end;
a tube extending from the proximal end to the distal end of the catheter; the tube further comprising:
a first curve and a secondary curve disposed between the proximal end and the distal end;
the first curve located proximate to the proximal end;
the secondary curve located distal to the first curve and disposed proximate to the distal end;
the secondary curve being configured in an opposite direction to the first curve;
an intermediate section disposed between the first curve and the second curve;
wherein the first curve has a first inner radius, a first arc angle, and a first arc length; and
the second curve has a second inner radius, a second arc angle, and a second arc length.

2. The method of claim 1, wherein the first inner radius is from about 0.10 cm to about 10.0 cm.

3. The method of claim 2, wherein the first inner radius is from about 1.0 cm to about 5.0 cm.

4. The method of claim 1, wherein the first arc angle is from about 10 degrees to about 170 degrees.

5. The method of claim 4, wherein the first arc angle is from about 30 degrees to about 150 degrees.

6. The method of claim 1, wherein the first arc length is from about 0.10 cm to about 30.0 cm.

7. The method of claim 6, wherein the first arc length is from about 0.75 cm to about 20.0 cm.

8. The method of claim 1, wherein the second inner radius is from about 0.10 cm to about 10.0 cm.

9. The method of claim 8, wherein the second inner radius is from about 0.5 cm to about 5.0 cm.

10. The method of claim 1, wherein the second arc angle is from about 10 degrees to about 170 degrees.

11. The method of claim 10, wherein the second arc angle is from about 30 degrees to about 150 degrees.

12. The method of claim 1, wherein the second arc length is from about 0.10 cm to about 30.0 cm.

13. The method of claim 12, wherein the second arc length is from about 0.20 cm to about 20.0 cm.

14. The method of claim 1, wherein the distal end of the catheter is attached with a functional device.

15. The method of claim 14, wherein the functional device comprises a medical balloon, a stent, balloon tip, end-hole catheter, closed-tip catheter, or a combination thereof.

16. The method of claim 1, wherein the S-Shape catheter is either pre-manufactured or is formed by a practitioner before the operation of inserting the catheter.
